(19) United States
(12) Patent Application Publication
(10) Pub. No.: US 2006/0251727 A1
(43) Pub. Date: Nov. 9, 2006

(54) Supplemental dietary composition for increasing muscle size, strength, athletic performance and/or exercise capacity

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/399,885

(22) Filed: Apr. 7, 2006

Related U.S. Application Data
(60) Provisional application No. 60/670,424, filed on Apr. 11, 2005.
(60) Provisional application No. 60/776,325, filed on Feb. 23, 2006.

(51) Int. Cl. A61K 31/385; A61K 31/22; A61K 9/14

(52) U.S. Cl. 424/489; 514/440; 514/551; 977/906

(57) ABSTRACT

The present invention provides for a diet supplement comprising creatine ethyl ester HCl or derivatives thereof, creatine alpha-ketoglutarate, and alpha lipoic acid, wherein the supplemental dietary composition increases muscle size, strength, athletic performance and/or exercise capacity. Methods of using and producing the composition are also disclosed. The supplemental dietary composition may be suitable for persons seeking to increase muscle size and strength, and those seeking to improve athletic performance and exercise capacity.

### RELATED APPLICATIONS

The application is related to and claims benefit of priority to U.S. Provisional Patent Application Ser. No. 60/670,424 entitled “Supplemental Dietary Composition for Increasing Muscle Size, Strength, Athletic Performance and/or Exercise Capacity,” filed Apr. 11, 2005, the disclosure of which is hereby fully incorporated by reference. The application also claims benefit of priority of U.S. Provisional Patent Application Ser. No. 60/776,325 entitled “Compositions and method for increasing bioavailability of compositions for performance improvement”, file Feb. 23, 2006, the disclosure of which is hereby fully incorporated by reference.

### FIELD OF THE INVENTION

The present invention relates to a nutritional supplement including creatine ethyl ester HCl, and on consumption increases muscle size, strength, athletic performance and/or exercise capacity.

### SUMMARY OF THE INVENTION

The present invention provides for a diet supplement comprising about 91% creatine ethyl ester HCl or derivatives thereof, about 4.5% creatine alpha-ketoglutarate, and about 4.5% alpha lipoic acid, wherein the supplemental dietary composition increases muscle size, strength, athletic performance and/or exercise capacity.

The present invention also provides, by the consumption of the supplemental composition, a method of increasing muscle size, strength, athletic performance and/or exercise capacity.

### DETAILED DESCRIPTION OF THE INVENTION

The present invention, according to various embodiments thereof, is directed to a diet supplement that includes creatine ethyl ester HCl. According to various embodiments of the present invention, the diet supplement may also include one or more of alpha lipoic acid and creatine alpha ketoglutarate. The diet supplement according to this invention provides a method of increasing muscle size, strength, athletic performance and/or exercise capacity.

According to various embodiments of the present invention, the diet supplement may be consumed in any form. For instance, the dosage form of the diet supplement may be provided as, e.g., a powder beverage mix, a liquid beverage, a ready-to-eat bar or drink product, a capsule, a tablet, a caplet, as a dietary gel, or in a container containing two compartments.

Furthermore, the dosage form of the diet supplement in accordance with this embodiment may be provided in accordance with customary processing techniques for herbal and/or dietary supplements in any of the forms mentioned above.

In one embodiment of the present invention, which is set forth in greater detail in Example 1 below, a diet supplement is provided for increasing muscle size, strength, athletic performance and/or exercise capacity. In this manner, the consumption of the supplemental composition provides for a method for increasing muscle size, strength, athletic performance and/or exercise capacity. According to the example embodiments set forth below, the diet supplement may be provided and consumed in any form, e.g., a powder beverage mix, a liquid beverage, a ready-to-eat bar or drink product, a capsule, a tablet, a caplet, as a dietary gel, or a container containing two compartments.

Furthermore, the dosage form of the diet supplement, in accordance with the example embodiment set forth below, may be provided in accordance with customary processing techniques for herbal and/or dietary supplements, wherein the active ingredients are suitably processed into a desired form. Those of skill in the art will appreciate that the diet supplement set forth in the example embodiment below may contain a variety of, and any number of different, excipients. For example, in one embodiment of the present invention, the diet supplement includes cellulose, calcium carbonate, croscarmellose sodium, stearic acid, magnesium stearate, silica and film coating (partially hydrolyzed polyvinyl alcohol, polyethylene glycol, titanium dioxide, talc, and FD&C Yellow NO.5).

Alpha Lipoic Acid

As used herein, “alpha lipoic acid” preferably refers to the chemical compound 1, 2-dithiolane-3-pentanoic acid, CAS registry No. 62464, also known as, Thioctic acid and 6,8-dithio octanoic acid. As used herein, “alpha lipoic acid” also includes derivatives of alpha lipoic acid such as esters, and amides, as well as other derivatives, including derivatives that become active upon metabolism. The chemical structure of alpha lipoic acid is as follows:

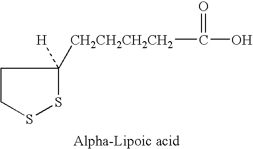

Alpha lipoic acid is an insulin modulator and an antioxidant that serves as protection against oxidative injury in non-neuronal and neuronal tissue (Bloch-Damti A, Bashan N. Proposed mechanisms for the induction of insulin resistance by oxidative stress. Antioxid Redox Signal. November-December 2005;7(11-12):1553-67. Review.) Alpha lipoic acid is a nutrient that the human body makes in minute quantities and may be obtained from yeast and liver (Wollin S D, Jones P J. Alpha-lipoic acid and cardiovascular disease.J Nutr. November 2003;133(11):3327-30. Review.) Studies have shown that alpha lipoic acid can significantly increase the body's utilization of blood sugar in type 11 diabetics and that lipoic acid may increase the metabolic clearance rate of glucose. In Europe, alpha lipoic acid has been used as a substitute for insulin in the treatment of Type II diabetes (Jacob S, Henriksen E J, Schiemann A L, Simon I, Clancy D E, Tritschler H J, Jung W I, Augustin H J, Dietze G J. Enhancement of glucose disposal in patients with type 2 diabetes by alpha-lipoic acid. Arzneimittelforschung. August 1995;45(8):872-4.).

Although the present invention is not to be limited by any theoretical explanation, it is believed that insulin is a primary factor that stimulates glucose and creatine transport into the muscle cells and that alpha lipoic acid both mimics and increases the actions of insulin in glucose and creatine transport into the muscle cells.

For example, the nutritional composition comprises between about 0.1 mg to about 1000 mg of alpha lipoic acid per serving of the nutritional composition. In an embodiment the nutritional composition comprises from about 0.5 mg to about 500 mg of alpha lipoic acid per serving of nutritional composition. In a further embodiment, the nutritional composition comprises about 100 mg of alpha lipoic acid per serving of nutritional composition.

Creatine

During digestion, creatine ethyl ester HCl is expected to rapidly and completely dissociate into creatine ethyl ester and hydrochloric acid in the acidic environment of the stomach. Following dissociation, hydrochloric acid is expected to combine with the hydrochloric acid already present as a component of gastric juice in the stomach. Since, creatine ethyl ester HCl is created by bonding an ethyl group to the carboxyl group of creatine through an esterification reaction, this ester bond is expected to be hydrolyzed in the small intestine resulting in the formation of ethyl alcohol (ethanol) and creatine. The portion of creatine ethyl ester molecules not hydrolyzed in the gastrointestinal tract will be transported to the liver, where hydrolysis of the ester bond will be carried out by β-esterase enzymes. In either case, creatine ethyl ester HCl is expected to be completely hydrolyzed into ethanol and creatine before it becomes bioavailable to tissues.

Creatine refers to the chemical compound N-methyl-N-guanyl glycine, CAS Registry No. 57-00-1, also known as, (α-methyl guanido) acetic acid, N (aminoiminomethyl)-N-glycine, and methylglycocyamine, and Methylguanidoacetic acid, and N-Methyl-N-guanylglycine. As used herein, the term “creatine” also includes derivatives of creatine such as esters, and amides, and salts, as well as other derivatives, including derivatives that become active upon metabolism.

Creatine is known to be present in the muscles of vertebrates. It is present in both phosphorylated and a non-phosphorylated forms and it is involved in muscular contraction and the development of fatigue by acting as a phosphate donor to ADP (Groff J L, Gropper S S. Advanced Nutrition and Human Metabolism. 3^{rd}. Ed. Wadsworth Thomson Learning. Scarborough, ON, 1999). Creatine is produced naturally by the body, but can also be obtained from animal foods.

Although the present invention is not to be limited by any theoretical explanation, it is believed that creatine increases strength, energy and muscle size as well as cell volumization and hydration. It is believed that increasing the amount of creatine within muscle increases muscular performance and the amount of work that can be done by the muscle. It has been widely reported that elevating the muscle total creatine store can increase performance during high-intensity exercise.

In accordance with one embodiment of the present invention, the creatine employed herein includes creatine ethyl ester HCl. Although the present invention is not to be limited by any theoretical explanation, it is believed that creatine ethyl ester HCl provides improved absorption and therefore increased bioavailability in the muscle. For example, the nutritional composition comprises between about 0.1 grams to about 10 grams of creatine ethyl ester HCl per serving of the nutritional composition. In an embodiment, the nutritional composition comprises from about 1.0 gram to about 5 grams of creatine ethyl ester HCl per serving of nutritional composition. In a further embodiment, the nutritional composition comprises about 2.1 grams of creatine ethyl ester HCl per serving of nutritional composition.

In accordance with one embodiment of the present invention, the creatine employed herein may also include creatine alpha-ketoglutarate. For example, the nutritional composition comprises between about 0.1 mg to about 1000 mg of creatine alpha-ketoglutarate per serving of the nutritional composition. In an embodiment, the nutritional composition comprises from about 0.5 mg to about 500 mg of creatine alpha-ketoglutarate per serving of nutritional composition. In a further embodiment, the nutritional composition comprises about 100 mg of creatine alpha-ketoglutarate per serving of nutritional composition.

The benefits to muscle size, strength, athletic performance and exercise capacity provided by the combination of ingredients set forth hereinabove is described in further detail in “Effect of Alpha-Lipoic Acid on Combined With Creatine Monohydrate on human Skeletal Muscle Creatine and Phosphagen Concentration,” Burke et al., International Journal of Sports Nutrition and Exercise Metabolism, 2003, 13, 294-302 (2003), the disclosure of which is hereby fully incorporated by reference.

As set forth above, the dosage form of the diet supplement, in accordance with the example embodiments set forth below, may be provided in accordance with customary processing techniques for herbal and/or dietary supplements, wherein the active ingredients are suitably processed into a desired form. In accordance with one embodiment of the present invention, one or more ingredients of the diet supplement are processed so as to form fine-milled particles. For instance, in one embodiment, one or more ingredients of the supplemental dietary composition is processed by a large-scale dry milling technique that produces fine particles, preferably known as fine-milled particles. The use of dry milling techniques, in combination with excipients and polymers, to form fine-milled particles has been shown to improve flow and dispersability, stability, resistance to moisture, bioavailability, and dissolution/release properties. Formulations benefit by containing fine-milled particles for the purpose of providing the one or more ingredients in particle sizes that optimize one or more of the flow and dispersability, stability, resistance to moisture, bioavailability, and dissolution/release properties of the one or more ingredients in a diet supplement. In vitro tests designed to simulate the environment of stomach were preformed to test the dissolution rate of fine-milled particle tablets versus non-fine-milled. These test showed that in tablets produced from fine-milled particles the time to 100% dissolution was approximately 15 minutes. In the case of non-fine-milled particle compositions, only 90% dissolution was observed after 120 minutes. In an embodiment, the supplemental dietary composition contains fine-milled particles having an average size between about 2 nm and about 50 nm.

U.S. Provisional Patent Application 60/776,325 discloses a method for improving the absorption, palatability, taste, texture, and bioavailability of compounds by increasing the solubility of compounds in proprietary formulations for the purposes of enhancing or improving muscle size, growth and/or recovery time and/or weight loss. The increased bioavailability of the compound or ingredients is achieved by reducing the particle size via “fine-milling” thereby increasing the surface area-to-volume ratio each particle, thus increasing the rate of dissolution. The compositions and methods disclosed promote increased bioavailability by increasing the total surface area of poorly soluble particles, thereby increasing the rate of absorption.

As used herein, the term “fine-milled” and/or “fine-milling” refers to the process of micronization. Micronization is a mechanical process that involves the application of force to a particle, thereby resulting in a reduction in the size of the particle. The force, in the case of micronization, may be applied in any manner such as, e.g., the collision of particles at high rates of speed, grinding, or by an air-jet micronizer. In an embodiment, fine-milled particles are obtained by jet-milling with nitrogen and compressed air.

As used herein, the term “particle size” refers to the diameter of the particle. The term “average particle size=38 means that at least 50% of the particles in a sample will have the specified particle size. In an embodiment, at least 80% of the particles in a sample will have the specified particle size. In another embodiment, at least 90% of the particles in a given sample will have the specified particle size.

The size of a particle can be determined by any of the methods known within the art. Methods for particle size determination which may be employed are for example, e.g., sieves, sedimentation, electrozone sensing (Coulter counter), microscopy, and/or Low Angle Laser Light Scattering. The preferred methods for the particle size determination of the present invention are the methods which are most commonly used in the pharmaceutical industry, such as laser diffraction, e.g., via light scattering Coulter Delsa 440SX.

The fine-milling process may be employed in the processing of one or more of the ingredients of the present invention in the dosage forms of tablets, e.g., immediate-release film coated, modified-release and fast-dissolving; capsules, e.g., immediate-release and modified-release; liquid dispersions; powders; drink mixes, etc.

According to an embodiment of the present invention, the creatine ethyl ester HCl is fine-milled. Advantageously, at least from about 10% to about 40% of the creatine ethyl ester HCl is fine-milled, e.g., to between about 2 nm and about 50 nm in average particle size. Additionally or alternatively, according to an embodiment of the present invention, the alpha lipoic acid is fine-milled. Advantageously, at least from about 1% to about 20% of the alpha lipoic acid is fine-milled, e.g., to between about 2 nm and about 50 nm in average particle size.

Although the following example illustrates the practice of the present invention in one of its embodiments, the example should not be construed as limiting the scope of the invention. Other embodiments will be apparent to one skilled in the art from consideration of the specification of the following example.

### EXAMPLE 1

As an example, the following composition is provided: creatine ethyl ester HCl (2.1 g), creatine alpha-ketoglutarate (100 mg) and alpha lipoic acid (100 mg). Preferably, the nutritional composition is consumed daily, taking one serving in morning and one serving in the evening.

What is claimed:

1. A composition comprising about 91% creatine ethyl ester HCl or derivatives thereof, about 4.5% creatine alpha-ketoglutarate, and about 4.5% alpha lipoic acid.

2. A composition of claim 1, wherein the creatine ethyl ester HCl is fine-milled.

3. A composition of claim 1, wherein at least from about 10% to about 40% of the creatine ethyl ester HCl is fine-milled.

4. A composition of claim 3, wherein the creatine ethyl ester HCl is fine-milled between 2 nm and 50 nm in average particle size.

5. A composition of claim 1, wherein the alpha lipoic acid is fine-milled.

6. A composition of claim 1, wherein at least from about 1% to about 20% of the alpha lipoic acid is fine-milled.

7. A composition of claim 6, wherein the alpha lipoic acid is fine-milled between 2 nm and 50 nm in average particle size.
